(19) United States
(12) Patent Application Publication
(10) Pub. No.: US 2007/0095647 A1
(43) Pub. Date: May 3, 2007

(54) Method and apparatus for producing reactive oxidizing species

(76) Inventor: Dwayne Dundore, Clermont, FL (US)

Correspondence Address: David W Barman, 1101 NE 176 Terrace, North Miami Beach, FL, 33162, US

(21) Appl. No.: 11/263,200

(22) Filed: Oct. 31, 2005

(51) Int. Cl. C07C 1/00; B01J 19/12

(52) U.S. Cl. 204/157.15; 422/186.3

(57) ABSTRACT

A method and apparatus for producing Reactive Oxidizing Species (ROS) used to purify air, soil, and water is described.

### BACKGROUND OF THE INVENTION

Reactive Oxidizing Species (ROS) may be used to purify air, soil, and water.

Production of ROS and ROS Water

ROS is the term used to describe the highly activated air that results from exposure of ambient humid air to ultraviolet light. Light in the ultraviolet range emits photons at a frequency that when absorbed has sufficient energy to break chemical bonds. UV light at wavelengths of 250-255 nm is routinely used as a biocide. Light at 182-187 nm is competitive with corona discharge in its ability to produce ozone. Ozonation and UV radiation are both being used for disinfection in community water systems. Ozone is currently being used to treat industrial wastewater and cooling towers.

The combination of the two wavelength ranges (250-255 nm and 182-187 nm) is used to produce ROS. The ozone (O₃) produced at the higher wavelength is split into singlet oxygen (O⁻) by the lower wavelength range light. In the presence of water or humid air, singlet oxygen reacts with water to form hydroxyl radicals (OH⁻), hydrogen peroxide, (H₂O₂) and other reactive species. Using this combination of reactions, very little ozone is detectable. Instead, the major component of ROS is the hydroxyl radical, which has a higher activity than ozone. In the presence of other chemicals photolysis and photo degradation can produce a multitude of reactive species, including the chloride ion (Cl⁻).

ROS (ROS) has a multitude of uses: when it is dissolved in water it is very effective at eliminating bacteria and mold, enhances the power of detergents, can be used in many forms of pollution control, air, water, groundwater, and soil to remove organic compounds (VOCs). ROS is also effective in removal of sulfur oxides, SOₓ, and nitrogen oxides, NOₓ. The following table illustrates the effectiveness of ROS as it has been used in specific industries.

| INDUSTRY | PROCESSES | RESULT |
|---|---|---|
| Aqua Culture | Biocide | Cleaner tanks |
| (Includes: Fish farming, | Oxygen enhancement | Increased growth rates |
| eel farming, fresh and salt | | Increased productivity |
| water) | | |
| Automotive* | Air Abatement | Greater efficiency |
| | Water Treatment | Lower Costs |
| Commercial Laundry* | Water Treatment | Reduction of chemical use |
| | Odor elimination | Lower sewage costs |
| | Water Recycling | |
| Plating and Plastics | Water Purification | Improved product quality |
| | | Water reuse |
| Textiles | Water Treatment | Reduction of sewer costs and |
| | Indoor Air Improvement | haul off |
| Wood Finishing* | Air Treatment | Formaldehyde oxidation |
| Wood Products* | Retrofit Abatement | Increase efficiency |
| (Includes: panel, paper, | Equipment | Decrease emissions |
| chip board, OSB, etc.) | NOₓ Reduction | |

*In all of these industries, ROS technology has contributed to environmental compliance and reduction of fines.

Additional applications of this technology could cover many other industries and many other processes within the listed industries. In water remediation, it is often necessary to add chemicals to aid in the process of contaminant removal. ROS can replace or enhance the activity of many of these additives. It decreases the surface tension of the water changing its properties as a solvent, and in the process causes the suspended and dissolved solids to precipitate. Activated water, or water that has dissolved ROS, reacts with contaminant compounds including salts, fats, oils, greases, hydrocarbons, etc., changing their solubility, reactivity, and binding capabilities. It has been demonstrated that ROS treatment of contaminated water will lower the COD, BOD, suspended solids, and dissolved solids in the water.

ROS in Water

The uses of ROS in water as activated water are numerous. The concentration of activated species in water can be controlled and monitored thus allowing use as oxygen enhanced system and a biocide at the same time. This has been effectively demonstrated in fish farms and aquariums. The added oxygen in the water has increased the growth rate of fish while inhibiting the growth of algae, parasites, and bacteria. This biocide capability has also been shown effective in industrial areas where circulating and stagnant pools of water are necessary. The introduction of ROS into these types of water streams eliminates odor, bacterial growth and aids in the destruction of contaminant organic compounds.

Introduction of ROS to water containing dissolved solids or dissolved VOCs will cause oxidation of those species. Fats and oils when oxidized form esters which in a caustic atmosphere (high pH) will form soaps. Introduction of ROS also effectively alters the bonding properties of water changing the highly structured order by interrupting the hydrogen bonding capabilities; this lowers the surface tension of the water and its ability to hold the dissolved constituents. After complete sparging with ROS, many of the previously dissolved solids can be filtered out leaving cleaner water that can be reintroduced into the original process, used safely for irrigation, or disposed of in a sanitary sewer. ROS may be introduced before, during, or after suspended solid removal processes, the sequence is determined by the nature of the contaminants.

### SUMMARY OF THE INVENTION

In one embodiment, the present invention comprises:

A method of producing ROS comprising the steps of:
(a) Introducing ambient humid air into an environment whereby it may undergo a photolytic reaction;
(b) exposing ambient humid air to ultraviolet light in the presence of a metal catalyst;
(c) directing ambient humid air which has been exposed to ultraviolet light in the presence of a metal catalyst to an environment where it may be used as ROS.

In certain embodiments, the method further comprises said ROS used as a biocide, indoor air treatment, water purifier, mold eliminator, bacteria eliminator, eliminator of volatile organic compounds, eliminator of sulfur oxides, and eliminator of nitrogen oxide. The ROS method may produce hydroxyl radicals sufficient to carry out a desired purification process. This sufficient amount would be known by those skilled in the art and would vary depending on the solid, liquid, or gas to be purified and the nature of a particular purification.

In one embodiment, the method of the invention requires that the percent humidity is regulated within the range of 10-99%.

In another embodiment, the method of provides exposing ambient humid air to ultraviolet light in the presence of a metal catalyst occurs by application of metal catalyst to a fixed substrate in a method comprising the steps of:
(a) Preparing a mixture of polyvinyl chloride (PVC) with a metal catalyst and a solvent;
(b) Applying said mixture to a substrate;
(c) Heating said metal catalyst mixture to a temperature between 70-140° C.;
(d) Maintaining heat until solvent evaporated; and
(e) Placing said substrate in an apparatus where it may be exposed to UV light.

In one embodiment, of the method, has a metal catalyst that is titanium dioxide. The titanium dioxide may be in a concentration in the mixture of up to 90%, preferably 20-90%, and even more preferably, 50-87%.

Another embodiment of the present invention provides for an apparatus for producing ROS comprising:
(a) an inlet air means;
(b) a source of ultraviolet light;
(c) a metal catalyst; and
(d) an exit air means.

The inlet air means may be a fan or any other suitable means for introducing air into the apparatus. The ultraviolet light source produces at least one range of wavelength. In a preferred embodiment, the ultraviolet light produces more than one range of wavelength, even more preferred, the ultraviolet light source provides ultraviolet light in the ranges of 250-255 nm and 182-187 nm.

In a preferred embodiment, the apparatus has a metal catalyst comprising titanium dioxide, copper, copper oxide, zinc, zinc oxide, iron, and iron oxide or mixtures thereof, and more preferably, the metal catalyst is titanium dioxide.

In one embodiment, the apparatus of comprises a metal catalyst that is applied to a fixed substrate incorporated into said apparatus in a method comprising the steps of:
(a) Preparing a mixture of polyvinyl chloride (PVC) with a metal catalyst and a solvent;
(b) Applying said mixture to a substrate;
(c) Heating said metal catalyst mixture to a temperature between 70-140° C.;
(d) Maintaining heat until solvent evaporated;
(e) Placing said substrate in an apparatus where it may be exposed to UV light.

The apparatus may have an exit air means as an exhaust port. The exhaust port may further connect to a house for introduction of ROS into water by sparging. The apparatus may further comprising a source of humid air and said source of humid air is a spray nozzle providing atomized droplets of water.

The apparatus may further comprise a means to restrict the air flow and increase retention time in the unit.

The apparatus of may further comprise filtration means suitable for the coalescence of water.

The subject invention also includes a method for producing a substrate, coated with a metal catalyst, suitable for use in and apparatus for producing ROS comprising the steps of:
(a) Preparing a mixture of polyvinyl chloride (PVC) with a metal catalyst and a solvent;
(b) Applying said mixture to a substrate;
(c) Heating said metal catalyst mixture to a temperature between 70-150° C.;
(d) Maintaining heat until solvent evaporated;

The method includes metal catalyst comprising titanium dioxide, copper, copper oxide, zinc, zinc oxide, iron, and iron oxide or mixtures thereof, most preferable, titanium dioxide.

The solvent used in the method may be aqueous, organic, or a co-solvent mixture of aqueous and organic solvents.

The method further comprises metal catalyst in said mixture of polyvinyl chloride (PVC) with a metal catalyst and a solvent is present in said mixture at up to 90% by weight, preferably, 20-90%, and most preferably, 50-87%.

### BRIEF DESCRIPTION OF THE DRAWINGS

FIG. 1 shows the core of an apparatus for producing ROS

FIG. 2 depicts a cross section of the core of an apparatus for producing ROS

### DETAILED DESCRIPTION OF THE PREFERRED EMBODIMENTS

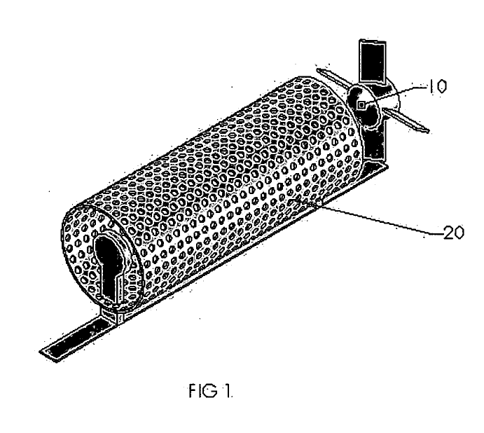

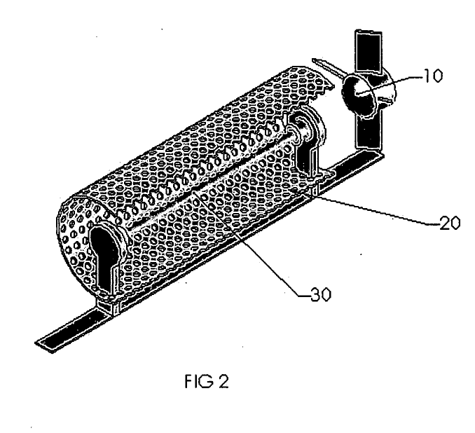

ROS, in one embodiment is produced when ambient air is exposed to ultraviolet light in the presence of a metal catalyst. In FIG. 1, The core of the apparatus is a substrate 20 in which metal catalyst has been deposited. FIG. 2 shows the apparatus whereby an inlet fan 10 draws ambient air into the central interior portion such that a beam of ultraviolet light 30 reacts with the ambient air in the presence on a metal catalyst that has been deposited on substrate 20. The ROS produced may be used for a wide variety of purification applications.

UV Cold Oxidation (water) Goal: To Remove Organics and Dissolved Solids from Water

Theory: Activated species (hydroxyl ions, radicals, hydoperoxides) produced from irradiation of ambient air is used to cause a breakdown of hydrocarbons in contaminated water.

Efficiency: Dependent on the content of the water and the time of ROS exposure, destruction efficiency can be as much as 95%.
Advantages: Removes dissolved solids from water
Disadvantages: Trial and error method of determining length of time and sequence of process, no recovery of organics.

The method used to introduce air into water is called sparging. Sparging produces very small air bubbles, which provides maximum surface area available for reaction. This is dynamically very similar to the fine spray of water being introduced into an air stream. There is a minimum size of particle that can be deposited by impaction on each size of droplet for any particular relative velocity of particle and droplet. Increasing the relative velocity or decreasing the droplet size reduces the minimum impactible particle size. There is a minimum particle size because a boundary layer of air adheres to the surface of the droplet. Small particles of limited velocity simply do not have enough inertia to penetrate the boundary layer. The minimum particle size is decreased when the droplet size is reduced because of decrease in the curvature radius of the gas streamlines as they bend around the droplet. This causes the particles to approach the droplets more closely before starting to cross streamlines and gives them a better chance of penetrating to the droplet surface. A particle can be deposited on a droplet by Brownian motion if it is small enough and has enough time to migrate to the droplet. This particle-separating force, known as diffusion, is enhanced by using the smallest possible target droplets because they most rapidly assume the velocity of the gas and this prolongs the available migration time. Using the smallest target droplets also produces the largest amount of surface and the greatest number of droplets possible from a given amount of water; both factors favor collision by Brownian motion. Increasing amounts of energy are needed to produce decreasing droplets sizes. Some of the devices (for example, the high-pressure spray nozzles of a fog scrubber) that produce the highest relative velocities of droplets and gas also produce the smallest droplets. As the droplets gradually assume the velocity of the air stream, Brownian motion gradually replaces impaction as the removal mechanism. It is theorized that some particles are entrained in the trailing eddy and thereby deposited on the trailing portions of high-velocity droplets. This accounts for the increase in deposition rate over the theoretical combined rate of diffusion and impaction that often occurs.

The concentration of ROS necessary for a particular treatment is determined by the desired outcome. 130-305 ppb ROS is all that is required to prevent bacterial growth in tanks used for fish farming. Much higher levels are required to oxidize VOCs in soil remediation. The desired concentration is achieved by regulation of: the flow rates of both the water and the air; the size of the air bubbles being sparged into the water; the temperature and turbulence of the water. When sparging water with ROS it is important to note that ROS species have a higher solubility in water than molecular oxygen and the life of the free radicals is lengthened in a water medium. Both of these physical characteristics add to the increased effectiveness of oxidation/reduction reactions in a water medium. Water with dissolved ROS species exhibits enhanced solvent power toward hydrocarbons. Materials partially or completely dissolve into the homogeneous mixture of ROS and water. The concentration of oxygen in the reactive system is high and the contact between the reactants is intimate, thus making chain scission reactions of hydrocarbons much more active. The initiation of chain scission reactions is important in the breakdown of organic chemicals. Once this process has been initiated, the energy released from the reactions is sufficient to fuel the propagating steps. This chain reaction continues until the chemical has been completely oxidized or this process can transform materials that would otherwise be non-soluble in to soluble species that dissolve in water allowing the oxidation reactions to occur in that medium. The result is the production of a harmless mixture of carbon dioxide and water.

Water Purification

When contaminant level is low and the desired treatment is purification, the water can be passed in a thin film past the UV light or held for an extended period in a reactor tank where the UV bulb is protected by a quartz sheath. Water is directly illuminated, not just sparged with ROS ROS is being used as part of drinking water purification systems in many parts of the world. Water purification is an application where the use of metals can be very beneficial.

Ultraviolet light at these same wavelengths can be used to photolytically activate certain metals that will then act as catalysts in degradation reactions.

ROS in Air

Air can be washed with ROS by substituting ROS for air in conventional air or steam stripping methods. ROS can increase the efficiency of air and vapor steam extraction processes. Indoor air can also be treated with ROS; because the ozone in the air is too low to be detected, it does not cause a regulatory concern. The concentration of ROS must be controlled, however, because the oxidation reactions of the components of ROS can be just as damaging to living organisms as ozone. At low concentrations ROS can be very effective in removing odors caused by smoke, organic chemicals, mold bacteria, etc. At higher concentrations, ROS can be used effectively to decontaminate houses with smoke damage, boats with mold and mildew, cars with tobacco odor, etc. ROS can be introduced into air streams and exhaust ducts to degrade VOCs (volatile organic compounds). As with the water, introducing reactive chemicals initiates combustion reactions involving the contaminants and the activated species. Introduction of as little as 0.6% ROS by volume into an exhaust duct at a large wood processing facility has been documented to lower the VOC concentration by 30%, the NOₓ by 20% and the CO by 15%. Increasing the ratio of ROS to exhaust air will result in increased destruction efficiency. Increasing contact between the ROS and the contaminants increase destruction efficiency. The Advance Oxygen Unit is designed to maximize both the concentration of ROS and the contact between the ROS and contaminants. Other design features include:
1. Introduction of humidity into the exhaust. This gives the contaminants and the activated species a medium on which to react, the water also acts as a catalyst to many reactions
2. UV lights illuminate the Advance Oxygen Unit tunnel where the contaminated air flows, the UV photons can photolytically activate, cleave, and induce reactions of the contaminating chemicals
3. Desired retention time and air flow rate determines the dimensions of the tunnel
4. Physical constriction or device to induce mixing
5. Photolytically activated metal catalyst in air flow line
6. Filters that allow water to coalesce
7. Water reaction tanks that allow soluble contaminants to be oxidized

When contaminated air streams are exposed to ultraviolet light not only the air but also the contaminants themselves become activated. UV cold oxidation technology is based on the use of the photons emitted as ultraviolet radiation, which have enough energy to break the bonds in organic molecules. Bond cleavage is not limited to carbon-hydrogen bonds but includes the higher energy carbon-halogen, and carbon-carbon bonds. Most abatement systems using ultraviolet technology also incorporate the added catalytic activity of a metal oxide. There is much recent research into development of effective photo catalytic metals, titanium dioxide, being the most extensively studied and currently considered the most effective.

The Advance Oxygen Unit UV Cold Oxidation Abatement System is set up to maximize the oxidation of hydrocarbons (destruction of VOCs) at ambient temperatures. The air flow rate in the exhaust duct is decreased significantly in a tunnel due to a much larger cross sectional area than in the exhaust duct. The lower flow rate allows a longer retention time in the tunnel. The breakdown of contaminant chemicals is dependent on the length of time they are exposed to the ultraviolet radiation in the tunnel. It has been determined that most chemicals are broken down within eleven seconds inside the tunnel. The tunnel is maintained at a high humidity, approximately 90-95%. The humidity is in the form of an activated fog. Activated water (described in the previous section) is introduced in several stages as a 5 μm droplet spray by a bank of spray nozzles. All the benefits that have been previously discussed for water reactions will occur in this humid environment plus the added destruction capability of the UV light.

The longer the contaminants can be held in this illuminated tunnel the more thorough will be the oxidation. One way to hold the contaminants or slow the air stream is to introduce screens or filters that restrict airflow. If the screens are made out of a metal like titanium dioxide or some other photolytically activated metal the oxidation reactions will be catalyzed when the contaminants encounter the screen. (See discussion on photolytically activated metals).

The combined oxidation potential of the metal catalyst, UV radiation, and free radical processes provides an abatement alternative that has higher destruction efficiency than that of the RTO (Regenerative Thermal Oxidizer) or the RCO (Regenerative Catalytic Thermal Oxidizer). Another advantage over thermal technologies is lower operating costs. The UV tunnel operates at ambient temperatures and so requires significantly less energy to operate. Operating costs have been projected to be one third that of operating an RTO.

UV Cold Oxidation (Air) Goal: To Remove Organics and Other Chemicals from Air

Theory: The use of the oxygen activated species (hydroxyl ions and radicals, hydro-peroxides) produced from irradiation of ambient air to initiate breakdown of hydrocarbons in contaminated air.

Efficiency: 99% efficient in pilot tests
Advantages: Energy efficient, does not contribute to thermal pollution, will eliminate not add to NOₓ emissions, versatility
Disadvantages: Not thoroughly tested in the field

It has been adequately demonstrated that the hydrocarbons, methyl ethyl ketone, methyl amyl ketone and xylene (the main constituents of paint), are completely oxidized to CO₂ and H₂O within 11 seconds when exposed to ultraviolet light at between 180 and 270 nm in a UV cold oxidation tunnel. Systems of these types have been proven effective at General Dynamics and several other industrial applications on the west coast.

### Background and Theory

### Chemical and Physical Explanation of Observed Oxidation

Photolytic Reactions

Photochemistry and Photolysis

Photochemical reactions are highly specific and their products quite different from those of thermo chemical reaction processes. Photolysis is defined as the chemical decomposition of the radiated material. The photolysis of molecular oxygen produces singlet oxygen.
O₂+hγ→2O⁻

The first law of photochemistry (Draper-Grotthus) states the light that is absorbed causes chemical change. Planck's Law is the fundamental law of quantum theory, expressing the essential concept that energy transfers associated with radiation such as light or x-rays are made up of definite quanta or increments of energy proportional to the frequency of the corresponding radiation. This proportionality is usually expressed by the quantum formula
E=hγ

In which E is the value of the quantum in units of energy and γ is the frequency of the radiation. The constant of proportionality, h, is known as the elementary quantum of action or, more commonly, as the Planck constant.

Light is measured in quanta. The wavelength of the light can be related to the frequency by the Stark-Einstein Law E=Nhc/λ. Where
N=Avagadro's number
h=Planck's constant
c=velocity of light
λ=Wavelength of light
   Each molecule that takes part in a chemical reaction induced by exposure to light absorbs one quantum of radiation causing the reaction. The photolytic decomposition of chlorine proceeds as follows:
   Cl₂+hγ→2Cl⁻
   Cl⁻+H₂→HCl+H⁺
   H⁺+Cl₂→HCl+Cl⁻
   The cycle is stopped by Cl+Cl→Cl₂ and H+H→H₂ both of these “termination” reactions are slow compared to the others. The kinetics of these reactions result in just one quantum of light bringing about a combination of a million molecules of hydrogen and chloride.

Under the influence of light a molecule disassociates, each atom carries away one of the two electrons, which bonded the atoms together in the molecule. This symmetrical fission of a covalent bond is termed homolysis. The alternative dissociate process in which both electrons remain with one partner is heterolysis. Reactions of UV light in humid environment include both types, but predominantly homolysis. The ultraviolet light acting on air and the H₂O provided by the humid environment produces a multitude of active ions and free radicals.

Free Radical Reactions

Production of Free Radicals

UV radiation at wavelengths between 180 to 400 nm provides energy in the range of 72 to 155 kcal/mole. In the presence of air and either water or high humidity, these amounts of energy are quite ample for producing free radicals and other species in varying degrees of photochemical excitement. Examples are excited atomic oxygen species (O), hydroxyl radicals (HO*), hydroperoxy radicals (HO₂*), and ozone (O₃). Significantly, in the presence of high humidity or in aqueous solutions dissolved ozone immediately reduces to other radical forms and the dissolved ozone level goes to zero, yet the oxidative power of the solution is considerably higher than when ozone is present. The presence of water changes the effectiveness of other species also, for example, in water the super oxide anion (O^{●-}) is a better oxidant than O₂.

Comparative Oxidation Potentials

| Species | Volts |
|---|---|
| Fluorine | 3.0 |
| Hydroxyl Radical | 2.8 |
| Ozone | 2.1 |
| Hydrogen Peroxide | 1.8 |
| Potassium Permanganate | 1.7 |
| Hydrochlorous Acid | 1.5 |
| Chlorine Dioxide | 1.5 |
| Chlorine | 1.4 |
| Oxygen | 1.2 |

Many carbon-containing excited species are formed when hydrocarbons and other air contaminants come in content with the hydroxyls and peroxides. ROS will react with hydrocarbons to form organic free radicals.

Free Radical—term reserved for short-lived radicals possessing a magnetically non-compensated electron, or somewhat longer-lived, larger organic molecules of the aryl-alkyl type similarly possessing unpaired electrons in their valence shell. If a partial charge is developed, the reactivity of the system is enhanced. This enhancement increases with the weight of the charged form, which depends on the nature of the radical and of the substrate. This effect, known as the polar effect can appear in all types of radical reactions, such as hydrogen transfer, addition to double bonds, etc.

Hydrocarbons

Hydrocarbons comprise the largest group of contaminating compounds; all volatile organic compounds are hydrocarbon based. One of the most difficult bonds to break is the carbon-carbon bond in a hydrocarbon chain. The following sequence of oxidation reactions shows several possible pathways available for the combustion of a hydrocarbon in the presence of oxygen.

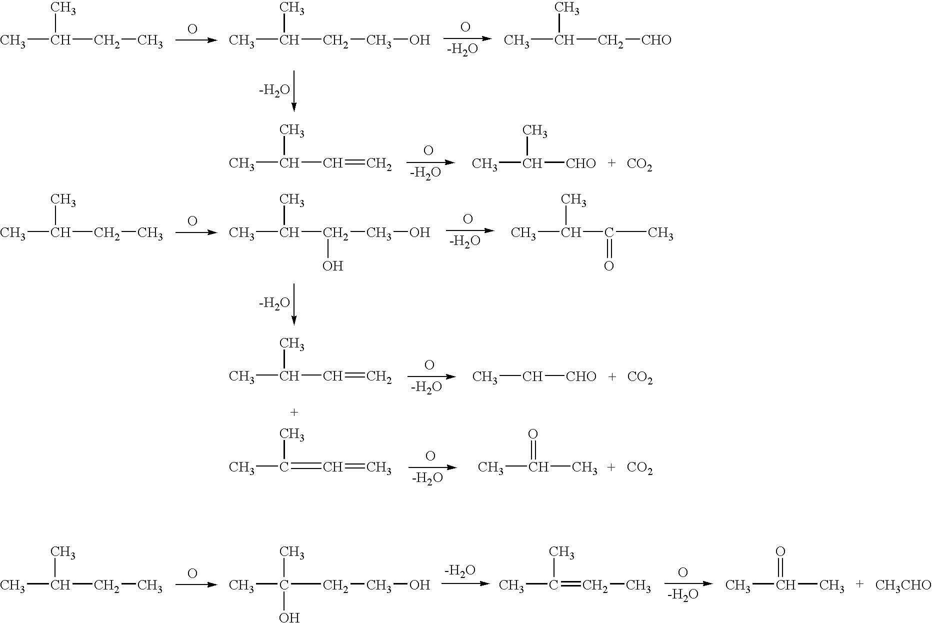

These reactions show how oxidation reactions produce a broad spectrum of partial oxidation products. Many of these products are active radicals capable of reacting with other compounds. The following table lists ionization potentials of some of the organic free radicals that can be produced when chemicals come in contact with UV lights or react with the radical species in A. O.

Ionization Potentials of Oxidants
Ionization potential is expressed in millivolts.

| | Ionization | | Ionization | | Ionization |
|---|---|---|---|---|---|
| Radical | Potential | Radical | Potential | Radical | Potential |
| •CH₃ | 9.84 | Ph• | 8.25 | •CH₂•CO₂H | 10.9 |
| 1-C₄H₉• | 8.02 | CH₂Ph• | 7.20 | O | 13.6 |
| 2-C₄H₉• | 7.25 | Cl• | 12.97 | H•C═O | 8.1 |
| t-C₄H₉• | 6.70 | •CCl₃ | 7.8 | •OH | 12.8 |
| cy- | 7.21 | MeO• | 8.6 | •OOH | 11.5 |
| C₄H₉• | | | | | |
| CH₃CO• | 7.9 | Cl₂ | 11.5 | O₂ | 12.1 |

Ionization potential represents the minimum amount of energy required to remove the least strongly bound electron from an ion or atom, this energy is expressed in electron volts. A high ionization potential means that the species can easily react or excite other compounds. The presence of species with high ionization potentials induces chemical reactions with many molecules.

Reactions, which proceed via radical intermediates, are often referred to as “homolytic reactions.” Free radical autoxidatiton is a largely indiscriminate process and gives high selectivites only with molecules containing one reactive site. Excited states of organic molecules can exist in singlet, doublet, and triplet states. The lifetimes of these states are very short, but with the detection capabilities of methods like flash photo lysis, the ability to detect these transient active species is now possible. When a molecule is in an excited state its reactions are often different from those it normally exhibits in its ground state and an organic compound often can exist in more than one excited state. The reactions of the excited states of the organic compounds are faster and more effective than the reactions compounds in there none cited states. The result is that more chemical breakdown is achieved.

In many cases, the oxidation of a volatile compound proceeds because of the energy released from the previous reaction. In this way, oxidation is self perpetuating and once set in motion will not stop without interference. The goal of abatement is to provide the energy to initiate these reactions and to maintain available energy to speed the breakdown to the thermodynamically favored end products CO₂ and H₂O.

Chain Reactions and Unit Steps

Chemical reactions involving radicals are called “chain reactions”. There are three categories of reactions in chain reactions: Initiation, Propagation, and Termination.

The following set of reactions is typical of hydrocarbon autoxidation—liquid-phase. Free-radical autoxidation of hydrocarbons by triplet molecular oxygen are numerous and well documented in the literature. The basic equations of the free-radical chain reactions of oxygen with hydrocarbons in which kinetic chains are long and hydro-peroxides are major primary products:

Initiation
X₂→X●
RH+X● (or XO₂●)→R●+XH (or XO₂H)
Propagation
R●O₂→RO₂●
RO₂●+RH→RO₂H+R●
Termination
2RO₂●→ROOR+O₂
(where X● is any radical and R is any hydrocarbon, alcohol, or carbonyl)
Energy necessary for the initiation step can be photolytic, electric, or chemical.

Aerial oxidations are usually difficult to initiate, but once underway they are often difficult to interrupt short of the most thermodynamically stable products, namely CO₂ and H₂O.

Chlorine

Chlorine atoms or radicals are not formed spontaneously at ambient temperature the reaction must be initiated. Under the influence of light, the chlorine molecule disassociates homolytically, thus producing two chlorine radicals. Any contaminant species containing chlorine will also liberate chloride ions when exposed to the UV light. Every Cl⁻ radical formed can split 116 O₂ molecules resulting in 232 O⁻ radicals.

The dehydrochlorination rate increases substantially in a wet oxidation environment and this acceleration is believed to be due to peroxy radicals, which are formed by the straight oxidation of a hydrocarbon or a fraction of polymer. This has yet to be confirmed experimentally.

Dehydrochlorination and Chain Scissions

Free-Radical Initiated Oxidation
—CH₂—CHCl—CH₂—CHCl—+ROO.—.CH═CH—CH₂—CHCl—+—Cl.→
—CH═CH—CH₂—CHCl—+—HCl+—ROOCl→—CH═CH—CH═CH—++O₂→
CO₂+H₂O
CH₂═CHCl+5+O₂→2CO₂+H₂O+HCl
An optimal process condition and time must be sought, especially in terms of the residence time to assure reactions would proceed to completion producing only H₂O, CO₂, and HCl. Products of incomplete combustion when they occur are usually in a form (like aldehydes or carboxylic acids) that can be rendered harmless by adjustment of pH and chemical additions that will form salts.
Solubilization

Solubilization plays an important role removal of contaminant compounds. The peroxides and hydroxyls in the ROS rob the hydrogen from carbon chains saturated with hydrogen's, forming double bonds in the hydrocarbon chains and react with the end methyl groups to form alcohols or carboxylic acids. The initially hydrophobic molecule is now more soluble. The solubility of a substance in water depends on its ability to form hydrogen bonds with the water. Polar solvents, such as water, consist of polar molecules that exert local electrical forces. Water has an unsymmetrical charge distribution caused by the highly electronegative oxygen atom polarizing the O—H bond. These forces result to give water an unusually high surface tension i.e. highly structured configuration in its liquid form. The higher the surface tension of water the lower its wetting ability, simply because the strong hydrogen bonding between the water molecules limits the bonding of water to other chemicals. One important property of ROS is that it reduces the surface tension of the water enhancing its wetting ability.

F.O.G.

The ROS species react with fats, oils and greases by adding oxygen to their structures. As these substances become more oxygenated, they become more soluble in water. When this process occurs at pH 10 or 11, the resulting substances are soaps, which we know are soluble or miscible in water.

It has long been known that soils are particularly sensitive to high pH. At a pH of 10 or 11 the reaction of ROS and F.O.G. are saponification reactions, which turn the fats into soaps.

A typical fat or oil structure involves a long hydrocarbon chain; these chains may be connected by glycerol forming triglycerides. The common structural features of fats, oil, and greases make them virtually insoluble in water and therefore, a particularly tough problem for abatement in a water medium. The following is an example of one possible breakdown reaction of a typical hydrocarbon that has a double bond at every third carbon. These allylic C—H bonds are relatively weak; the C—H bonds of the central methylene group in skipped dienes are “doubly allylic” and are especially weak. They are consequently unusually susceptible to radical attack. Any peroxyl radical will selectively abstract hydrogen from these doubly allylic sites, removal of a hydrogen atom results in a resonance-stabilized dienyl radical. 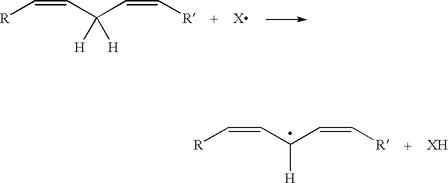
Alkyl radicals can then react further with the oxygen species. The double bonds in this example form upon further oxidation a carboxylic acid (carboxylate at pH 10). The more oxygenated these hydrocarbon chains become the more soluble they will be in water. Radical-chain oxidation therefore results in easier breakdown and solubilisation of these problematic molecules.

The formation of colloids in wastewater is a form of particle conditioning. If the wastewater has a high level of fats, oils and greases these reactions could result in the formation of micelles the core of a micelle has been shown to have properties similar to a liquid hydrocarbon. Any colloidal species of this sort that are formed aid in preventing contaminants from being re-entrained or re-dissolved.

Particulate

Particulate matter is susceptible to the reactivity of the ROS species if any part of the particulate can be oxidized. The enhanced wetting ability of the water caused by the ROS will wet the particulate. The bubbling action (sparging) of the ROS into the water provides the physical action to mix the water. The addition of ROS increases the wetting of the material by breaking the surface tension of the water. The dissolved negatively charged ROS species surround the positively charged particles with negative species. Both of these factors aid in the removal of particulate. Water with lower surface tension will cause suspended solid to drop out.

Salts

The presence of calcium and other salts in water may cause the formation of less dispersible residues with certain soils. Materials can be added to wastewater treatment processes that are designed to chelate the metal ions, particularly Ca⁺⁺ and Mg⁺⁺ ions. These chemicals sequester or precipitate metal ions in a colloid ally stable form. Several agents that are particularly effective at chelating calcium are sodium hexametaphosphate Na₆(PO₃)₆ and pentasodium tripolyphosphate. The hydroxyl ion in ROS will also react with the polyvalent metal ions, forming salts, which will precipitate.

The following is an example of a reaction that is photolytically initiated: Ketone-Hydrocarbon Reactions. The aliphatic ketones contribute to the reaction of hydrocarbons. The primary quantum yield for the photodissociation of acetone at 3130 ÅÅÅ has been reported as 0.9. Acetone dissociates as shown in the following equation:
CH₃COCH₃+hγ→CH₃CO●+CH₃●
→2CH₃●+CO
Acetone and diethylketone have been irradiated in the presence of 2-methly-1-butene and sunlight fluorescent lamps for periods ranging from 1 to 3 hr.

The chemistry of these reactions is enhanced by the presence of transition metal ions, which can catalyze hydro peroxide decomposition. A complete rationalization of observed effects of different metals is not completely understood but research in this area is continuing.

Metal Catalyzed Reactions

The presence of metals can be beneficial in remediation because of their catalytic capability. Metals, semi-metals, and semiconductors exist in a lattice formation of fixed positive ions immersed in a sea of conduction electrons, which are free to move through the lattice. Every direction of electron motion is equally probable, the main restriction on the movement or “freedom” of the electrons is the physical confines of the metal itself. The constant state of energy of the metal surface is called the Fermi level.

The position of the Fermi energy may be different under different situations such as when the oxide is in contact with another medium. When there are adsorbates on the oxide surface, or when the surface is partly reduced. These situations could result in a surface region of the solid that is very different from the bulk, and a potential gradient is developed between the surface and the bulk. Associated with this potential gradient is a distribution of densities of electron and holes by thousands or millions times.

When a sample is placed in water, which contains a redox couple A−/A, electron transfer between the solid, and the solution occurs until the electrochemical potential of the valence electrons in the solid equals that in the solution. If the redox couple A−/A is the only species that can exchange electrons with the solid. The electrochemical potential of the solution is defined by the standard redox potential of this couple and the relative concentrations of A⁻ and A. Before the solid contacts the solution, the Fermi energy of the solid is higher than the electrochemical energy of the electrons in the solution, and after contact, electrons flow out of the solid to the solution to establish equilibrium (that is, equalization of electrochemical potential) across the interface. The outflow of electrons from the solid results in a net positive charge near the surface in the solid, which eventually sets up a potential barrier to stop the electron flow. Equilibrium is then established. The region of the solid near the surface that has a net charge is called the depletion layer.

Photo-Activated Metals

Certain metals can be activated photolytically. The magnitude of the photo-effect depends on the oxide. For example, photo enhanced processes are readily observed on ZnO and TiO₂, but are much weaker on V₂O₅. This photo-enhanced catalytic oxidation is paralleled by photo-adsorption activities. The adsorption of oxygen on V₂O₅ does not show any response to light, and WO₃ shows only a weak response.

Photoelectric effect is the change in electrical characteristics of a substance due to radiation, generally in the form of light. Radiation of sufficiently high frequency (short wavelength), impinging on certain substances, particularly, but not exclusively, metals, causes bound electrons to be given off with a maximum velocity proportional to the frequency of the radiation, i.e., to the entire energy of the photon. The Einstein photoelectric law, first verified by Millikan, states: Eₖ=hy−ω where Eₖ is the maximum kinetic energy of an emitted electron, h is the Planck constant, γ is the frequency of the radiation (frequency of the absorbed photon), and ω is the energy necessary to remove the electron from the system, i.e., the photoelectric work function for the surface of the emitting substance. One of the principal aspects of the photoelectric effect is photoconductivity. Photoconductivity is the phenomenon evidenced by the increase in electrical conductivity of a material by the adsorption of light or other electromagnetic radiation. Relatively few materials give exhibit large changes of conductivity with illumination.

The most important characteristics of metal catalysts for effecting oxidation are the accessibility of several oxidation states as well as the accommodation of various coordination numbers. Both of which are properties of transition metal complexes. In the electronic band structure of the valence electrons of transition metal oxides the 2p orbital overlap to form a filled valence band, and the s and d orbital of the cations overlap to form the conduction band. The conduction band is at a higher energy than the valence band and separated from it for most transition metal oxides at their highest oxidation states. The electrochemical energy of the valence electrons in the solid is between the conduction band energy and the valence band energy. The density of electrons in the conduction band can be increased by incorporating donor ions in the solid that donate valence electrons to the conduction band, and the density of holes in the valence band can be increased by acceptor ions that accept electrons from the valence band.

When the photo-generated holes and electrons migrate to the surface, light-induced surface chemistry becomes noticeable. When the solid absorbs a photon of light of energy larger than the band gap, an electron is excited from the valence band to the conduction band. Because of the upward bending of the electron bands near the surface, the photo-generated hole tends to migrate to the surface, while the electron tends to migrate to the bulk. This results in an increased whole concentration at the surface, which facilitates surface reactions involving electron transfer to the solid, such as oxidation reactions. This situation persists as long as the solid is illuminated and there is a mechanism to remove the electron that has migrated to the bulk. Flow of electrons from the oxide via an external circuit to a counter electrode is one way to remove them.

If there is no mechanism to remove the electrons from the bulk, continuous illumination will result in accumulation of electrons and holes near the surface, to the extent that the bending of the electron bands is removed. The photo-generated electrons become available at the surface in significant concentrations, which facilitate surface reactions that involve transfer of electrons from the solid, such as reduction reactions.

Recently there has been a strong interest to replace the external circuit that connects the counter electrode and the irradiated oxide by direct contact of the two electrodes. Platinum deposited on TiO₂ is the most popular system that has been investigated. In this system, the photo-generated holes are consumed in the surface reaction at TiO₂. The photo-generated electrons migrate to platinum where they are consumed in a reaction at the platinum surface. The complete oxidation-reduction cycle can be accomplished on one composite particle.

The catalyst used in the Advance Oxygen Unit system was designed to utilize the photoelectric effect of the combination of metals. The Copper/Zinc metal combination (KDF®) provides a surface for the acceptance of electrons produced photolytically on the TiO₂ surface.

It is seen from this description that the photo-effect is significant only when a large concentration of photo-generated holes and/or electrons are present at the oxide surface. Since most of the light is absorbed by the region of an oxide up to a few hundred nanometers from the surface, these holes and electrons must have sufficiently long lifetimes to reach the surface. Therefore, reduction of an oxide and the presence of lattice defects and impurities, which shorten the lifetime of these holes and electrons, will reduce the photo-effect. The lifetime also depends on the effective masses of these carriers, which depend on the band structure of the solid.

A side effect that occurs on illumination is heating of the oxide. A majority of electrons and holes recombine nonradiatively. The energy released heats up the oxide. In many experiments, light sources are used that give a broad spectral distribution. Some light of energy less than the band gap energy is absorbed by the photon mode of the oxide and heats up the solid. The extent of heating depends on the oxide and the light source used. A temperature rise to 60° C. from room temperature has been reported. (This temperature is detected in the Advance Oxygen Unit tunnel.

Photo-assisted adsorption and desorption Absorption of light of energy larger than the band gap energy increases the surface concentrations of electrons and holes of a semi conducting oxide significantly and enhances rate processes such as adsorption and desorption that involve electron transfer.

Photo-assisted adsorption and desorption of oxygen is the most studied process in this category.
Adsorption: e⁻+O₂→O₂⁻ (ad)
Desorption: h⁺+O₂⁻ (ad)→O₂ (g)
Both adsorption and desorption can be enhanced by irradiation. Band gap illumination of ZnO at room temperature results in photo-assisted adsorption of oxygen at low pressures. The effect decreases with increasing pressure, and eventually becomes photo-assisted desorption at high pressures. At high temperatures, the effect of pressure is opposite.

Detection of photo-assisted adsorption can be made by following changes in the conductivity of the sample. Since such adsorption changes the concentration of charged species on the surface and consumes photoelectrons or holes, it changes the conductivity of the sample. It has been found that the surface returns only slowly to the equilibrium state after the light is turned off, thus the enhanced adsorption due to irradiation must be retained for a long time, and these species are practically irreversibly adsorbed.

The surface condition is important. It has been shown that the amount of oxygen photo-adsorbed on TiO₂ increases with the amount of hydroxyl groups on the surface. A similar observation has been reported that dehydroxylation of the surface reduces photo-adsorption of oxygen on TiO₂, which can be restored by rehydrating the surface. It is believed that hydroxyl groups are important in trapping photo-generated holes, making photoelectrons available for chemisorptions:
OH⁻+h⁺→.OH (ad)
In the presence of hydrocarbon molecules, the hydroxyl radicals participate in oxidation reactions. Photolysis of the lattice resulting in evolution of lattice oxygen has been reported on ZnO. Photodecomposition of TiO₂ has not been observed.

The photo-response of a sample depends on its pretreatment, especially if the pretreatment results in a slightly reduced or fully oxidized sample. Reduction usually leads to n-type semi conductivity, and the extent of reduction determines the density of conduction electrons and the lifetime of photo carriers. For some oxides, severe oxidation may lead to excess lattice oxygen and p-type conductivity. Coupled with the effect of surface hydroxylation, it is not surprising that there are conflicting reports in the literature on the effect of illumination. Doping of an oxide changes its semi conduction properties and response to irradiation. Addition of Li to ZnO enhances photo-adsorption of oxygen, while addition of Ga or Al reduces it.

Photo catalysis: Gas Phase Reactions a large number of gas phase catalytic reactions have been found to be greatly enhanced by illuminating an oxide with light of band gap energy.
1. oxidation of hydrocarbons
2. oxidation of alcohols
3. oxygen isotope exchange
4. oxidation of CO, NH₃
5. H₂-D₂ exchange
   In general, these reactions proceed readily at elevated temperatures in the dark. Thus, most of the studies of light enhancement have been conducted near room temperature. Product selectivity's are generally different between thermal and light-assisted reactions. Oxidation of hydrocarbons is among the most studied of reactions, particularly the oxidation of alkanes. Using TiO₂ as the catalyst, photo catalytic oxidation produces a wide range of products. Combustion is usually a significant reaction. Cracking of the carbon chain to products of lower carbon numbers is also a common process. The reactivity of various types of carbon atoms on TiO₂ follows the sequence:
   Cₜₑᵣₜ>C_{quat}>C_{sec}>Cₚᵣᵢₘ
   The reactivity does not seem to follow the ionization potential of the hydrocarbons.

Since the activity in the photo catalytic oxidation of hydrocarbons is proportional to the light intensity, and the reaction only proceeds in the presence of oxygen, it has been assumed that photo-adsorbed oxygen and photo-generated holes are both important. Photo catalytic oxidation of alcohols at room temperature in the presence of oxygen proceeds readily on a number of oxides. For example, the oxidation of 2-propanol on rutile (TiO₂) results almost exclusively in acetone. Continual exposure of the reaction mixture to illuminated TiO₂ results in further oxidation of acetone to formic acid, and eventually CO₂ and water. However, the rate of oxidation of acetone is slow.

Changes in the metals used to catalyze oxidation reactions can be optimized for the desired result. In pollution control, the choice of the catalyst used can be chosen to fit the contaminant load. In the case of the Advance Oxygen Unit, the composition of catalyst can be specially designed to promote the necessary oxidation-reduction reactions. The design is versatile so that it may be altered and adapted to a specific abatement problem.

There is an emphasis in research to develop processes that will selectively oxidize substrates to desired products. Excellent work has been documented on the use of oxygen in its active form and selected metals to aid in these selective processes. The solution to removal of pollutant substances is almost opposite in emphasis. The process involved must be designed to be highly nonspecific and the desired combustion or oxidation reactions must proceed completely to CO₂ and H₂O. The design of the ROS Air Tunnel (Advance Oxygen Unit) and Water Systems using ROS was intended to maximize oxidation potential. The presence of the UV light photolytically activates any chemical that can absorb light in the range of 180-300 nm. This encompasses most organic chemicals. The high humidity containing reactive radicals provides a medium on which degradation reactions can occur. The presence of a photolytically activated metal in the air path acts to catalyze oxidation reactions, lengthen the time contaminants are exposed to UV light by physical barrier, and produce an electrical energy, which also aid in destruction. Contaminating chemicals that are soluble are dissolved into the water that is removed from the air tunnel in several places by use of a filter medium that provides a place for the water to coalesce. This water is pumped to a series of reaction tanks where the water is continually sparged with ROS and held within four inches of a UV light for a time sufficient to oxidize all contaminants. This same water is returned to the air tunnel through the fog nozzles after filtration.

The following five sections are added to show ideas that are being considered for future direction of research and development for improvements in the Advance Oxygen Unit System.

Oxygen Isotope Exchange

Light enhanced oxygen isotope exchange reaction has been studied in detail over TiO₂ and ZnO. Both the homophasic exchange reaction:
¹⁶O₂ (g)+¹⁸O₂ (g)<=>2¹⁶O¹⁸O (g)
and the heterophasic exchange reaction:
¹⁸O₂ (g)+¹⁶O₂ (lattice)<=>¹⁶O¹⁸O (g)+¹⁸O₂ (lattice)
Proceed readily at room temperature under band gap illumination.
Both homophasic and heterophasic reaction occur simultaneously on an illuminated oxide. The photocatalytic activity of a partially dehydroxylated TiO₂ is higher than a fully hydroxylated sample.

Other photo-oxidation reactions compete with the photo catalytic oxygen isotope exchange reaction. In the presence of 2-methylpropane, oxygen isotope exchange is completely suppressed on TiO₂ until the oxidation of the hydrocarbon is complete. On ZnO, CO oxidation suppresses the oxygen isotope exchange reaction. These results suggest that these reactions either proceed on the same sites or involve a common intermediate. Indeed, on TiO₂, the activity per unit area for oxygen isotope exchange correlates with that for 2-methylpropane oxidation, and the participation of O⁻ (ad) in both reactions has been suggested.

The decomposition of NO produces N₂ as the product at low NO pressures, and N₂O at high NO pressures. Adsorbed oxygen is the other product at room temperature. Rutile TiO₂ doped with Fe or illuminated anatase TiO₂ are quite effective in the decomposition of ammonia.

Photocatalysis: Liquid Phase Reactions

In gas phase photo catalytic reactions, charged reaction intermediates must remain adsorbed on the surface. In the liquid phase, however, charged species can readily exist, thus it becomes possible to remove an electron from an anion in the solution to form a neutral species by a photo electrode, or inject an electron into a neutral species to form an ion. The resulting species are then free to further react in solution.

Photo-assisted oxidation has also been demonstrated at a TiO₂ electrode for hydroquinone, p-aminophenol, I⁻, Br⁻, Cl⁻, Fe²⁺, Ce³⁺, and CN⁻ ions. Many of these species are oxidized at potentials negative of their standard redox potential. The oxidation of water has been studied extensively. Earlier work has been on the formation of hydrogen peroxide. The reaction mechanism can be either:
H₂O+O⁻ (ad)→HOO⁻ (ad)+H (ad)
HOO⁻ (ad)+H⁺ (ad)→H₂O₂
or
H₂O+O⁻ (ad)→OH⁻ (ad)+OH (ad)
OH⁻ (ad)+OH (ad)→H₂O₂+e⁻ (lattice)
Recently the interest has been shifted to the photo-assisted electrochemical oxidation of water to oxygen. It has been discovered that band gap illumination of a TiO₂ anode substantially reduces the applied potential required for the evolution of oxygen. In the absence of other more readily reducible species, proton is reduced to hydrogen at the cathode when a sufficiently high potential is applied. Thus, the net reaction is the decomposition of water.
Reduction Reactions

Photo-assisted reduction of metal complexes to metallic particles deposited on the oxide can be achieved in a number of systems. The reduction is accompanied by oxidation of another species. For example, copper ions can be reduced to copper metal deposited on illuminated TiO₂ particles in water and oxygen is produced.

There are reports on the photo-assisted reduction of water by oxides. p-Type Fe₂O₃ has been successfully used with n-type Fe₂O₃ to decompose water. Water is reduced to H₂ at the p-type iron oxide.

Photocatalysis by Metal/Metal Oxide and Oxide/Oxide Composites

Some oxide particles of electrodes are quite efficient in effecting photo-assisted electron transfer. For an n-type semi conduction oxide, this has resulted in enhancement in many oxidation reactions. For an effective photo catalyst, every step of the catalytic cycle must proceed efficiently. This requirement may not be met by many oxides. For example, photo-oxidation on TiO₂ is commonly observed; but photo reduction on this oxide is much less known. Thus, electron transfer out of TiO₂ is more difficult than into the oxide. Therefore, attempts have been made to deposit metal or another oxide on a semi conducting oxide that will facilitate electron transfer in the difficult direction. Noble metals and RuO₂ have been used as deposits for this purpose.

Sometimes a composite of n- and p-type semi conducting oxides are used when a single oxide does not generate sufficient photo electrochemical driving force to complete a desired reaction. Then a composite of two appropriate semi-conducting oxides could be used to supply a combined photo electrochemical driving force. Successful decomposition of water using visible light has been achieved using such devices.

The reduction step is necessary to produce metallic Ni to form a NiO/Ni/SrTiO₃ interface in which Ni serves as an ohmic contact. The activity in NaOH is higher than in water, perhaps because hydrogen peroxide decomposes more efficiently in basic than neutral solutions.

Abiological Catalysis of Oxidations

There is a marked difference between the gas and liquid phases, and when abating air with ultra violet light the best of both phases can be obtained with high humidity, (90-95%) environment. Spray nozzles that produce a very small droplet size will provide a medium on which oxidation reactions can occur, provide the catalytic power of H₂O and particle conditioning.
Liquid Phase—generally involve free radical autoxidation mechanism and a minority of processes that involve direct oxidation of the substrate followed by reoxidation of the reduced metal catalyst with dioxygen
Gas Phase—generally involves the so-called Mars-van Krevelen mechanism i.e. direct oxidation of the hydrocarbon by an oxometal species followed by regeneration with dioxygen.
   How can we account for this marked difference between the gas and liquid phases?
   The free radical autoxidation in the liquid phase is easy, ubiquitous, and difficult to compete with. The concentrations of RH in the vicinity of the catalyst are much lower making radical chain processes less favorable in the gas phase.

Catalysis of the oxidation of a hydrocarbon substrate by can proceed by involving a classical free radical autoxidation mechanism or direct oxidation by a metal salt or direct activation by ³O₂. Formation of deoxygenates complex, ³O₂, precedes in liquid phase, and follows in gas phase the oxidation of the substrate by on ox metal complex.

In one embodiment, the catalyst of the present invention may be prepared as follows:

Using a piece of metal; application of the catalyst material is prepared by cleaning it using one or a combination of sand blasting, pressure washing, or chemical washing. After the metal (base) is prepped, the metal is coated with a catalyst material. Depending upon the application, the catalyst material is made up of different amounts and/or proportions of titanium dioxide, copper and zinc oxide and iron mixed with poly vinyl chloride and a solvent. The catalyst is applied to the base using an Airless sprayer, pressurized sprayer, roller, brush or the base is dipped in the catalyst solution. After the catalyst is applied the base it is baked or cured in an oven at 150° F. for 15-20 minutes to create the catalytic component. Sufficient time is allowed for cool down. The catalytic component can be fabricated into the required shape and size by rolling, cutting, or forming and installed into the equipment. 1) the catalyst is mixed with poly vinyl chloride (PVC) and solvent, it is then applied to the metal, then it is heated to 150 degrees, this bakes off the solvent allowing the PVC to dry and adhering the catalyst and the PVC to the metal.

2) some power coating uses PVC, but the difference is that power coating uses static electricity to hold the coating onto the metal surface, then it is heated to 450 degrees to allow the coating to melt and cover the surface.

3) There are two methods other companies use to apply catalyst to metal. The first is to mix the catalyst with a paint or coating, then apply it. The problem with this method is that the UV light brakes down the lasamers that hold the paint together, all of the unit I have ever saw or tried only last up to 2 years before the coating is gone. The second method is to mix the catalyst with water and submerge the metal into it, filling the small pours in the surface. This method only allow small amounts of catalyst to be applied to the metal and can be removed from the metal very easily.

4) White power coating resin has up to 20% titanium dioxide in it as the color.

In some embodiments, ROS may be generated by a Medium Pressure Mercury Arc (MPMA) Lamp

MPMA lamps emit not only ultraviolet light, but also visible height, and wavelengths in the infrared spectrum. In fact, all lamps emit approximately 20% ultraviolet light. 60% infrared light and 20% visible light It is therefore important that when selecting a lamp, output in the ultraviolet spectrum should be closely examined The ultraviolet spectral output is sometimes expressed graphically, showing the proportional output at the important ultraviolet wavelengths A graph of a typical MPMA lamp is shown below The broad spectrum of the MPMA lamp is selected for its functionality. Wavelengths of UV light are produced of varying wavelengths providing the following advantageous uses:
The direct DNA deactivation of bacteria and viruses
Energy sufficient to overcome the band gap of titania in the anatase phase allowing for I its use as a catalyst in the oxidation process
Energy to excite carbon bonds making them susceptible to oxidation
   Generally, the invention has been described in its preferred form or embodiment with some degree of particularity, it is to be understood that this description has been given only by way of example and that numerous changes in the details of construction, fabrication and use, including the combination and arrangement of parts, may be made without departing from the spirit and scope of the invention.

I claim:

1. A method of producing ROS comprising the steps of:
(a) Introducing ambient humid air into an environment whereby it may undergo a photolytic reaction;
(b) exposing ambient humid air to ultraviolet light in the presence of a metal catalyst;
(c) directing ambient humid air which has been exposed to ultraviolet light in the presence of a metal catalyst to an environment where it may be used as ROS.

2. The method of claim 1 wherein said ROS is used as a biocide, indoor air treatment, water purifier, mold eliminator, bacteria eliminator, eliminator of volatile organic compounds, eliminator of sulfur oxides, and eliminator of nitrogen oxide.

3. The method of claim 1 wherein said ROS method produces hydroxyl radicals.

4. The method of claim 1 wherein the percent humidity is regulated within the range of 10-99%.

5. The method of claim 1 wherein said exposing ambient humid air to ultraviolet light in the presence of a metal catalyst occurs by application of metal catalyst to a fixed substrate in a method comprising the steps of:
(a) Preparing a mixture of polyvinyl chloride (PVC) with a metal catalyst and a solvent;
(b) Applying said mixture to a substrate;
(c) Heating said metal catalyst mixture to a temperature between 70-140° C.;
(d) Maintaining heat until solvent evaporated;
(e) Placing said substrate in an apparatus where it may be exposed to UV light.

6. The method of claim 5 wherein said metal catalyst is titanium dioxide.

7. The method of claim 6 wherein said titanium dioxide is in a concentration in the mixture of up to 90%.

8. The method of claim 7 wherein said titanium dioxide is in a concentration in the mixture of 20-90%.

9. The method of claim 7 wherein said titanium dioxide is in a concentration in the mixture of 50-87%.

10. An Apparatus for producing ROS comprising:
(a) an inlet air means;
(b) a source of ultraviolet light;
(c) a metal catalyst;
(d) an exit air means.

11. The apparatus of claim 10 wherein said inlet air means is a fan.

12. The apparatus of claim 10 wherein said ultraviolet light source produces at least one range of wavelength.

13. The apparatus of claim 10 wherein said ultraviolet light produces more than one range of wavelength.

14. The apparatus of claim 10 wherein said ultraviolet source provides ultraviolet light in the ranges of 250-255 nm and 182-187 nm.

15. The apparatus of claim 10 wherein said ultraviolet source provides ultraviolet light in the ranges of 250-255 nm or 182-187 nm.

16. The apparatus of claim 10 wherein said metal catalyst comprises titanium dioxide, copper, copper oxide, zinc, zinc oxide, iron, and iron oxide or mixtures thereof.

17. The apparatus of claim 10 wherein said metal catalyst is titanium dioxide.

18. The apparatus of claim 10 wherein said metal catalyst is applied to a fixed substrate incorporated into said apparatus in a method comprising the steps of:
(a) Preparing a mixture of polyvinyl chloride (PVC) with a metal catalyst and a solvent;
(b) Applying said mixture to a substrate;
(c) Heating said metal catalyst mixture to a temperature between 70-140° C.;
(d) Maintaining heat until solvent evaporated;
(e) Placing said substrate in an apparatus where it may be exposed to UV light.

19. The apparatus of claim 10 wherein said exit air means is an exhaust port.

20. The apparatus of claim 19 wherein said exhaust port is further connects to a house for introduction of ROS into water by sparging.

21. The apparatus of claim 10 further comprising a source of humid air.

22. The apparatus of claim 21 wherein said source of humid air is a spray nozzle providing atomized droplets of water.

23. The apparatus of claim 10 further comprising a means to restrict the air flow and increase retention time in the unit.

24. The apparatus of claim 10 further comprising filtration means suitable for the coalescence of water.

25. A method for producing a substrate, coated with a metal catalyst, suitable for use in and apparatus for producing ROS comprising the steps of:
(a) Preparing a mixture of polyvinyl chloride (PVC) with a metal catalyst and a solvent;
(b) Applying said mixture to a substrate;
(c) Heating said metal catalyst mixture to a temperature between 70-150° C.;
(d) Maintaining heat until solvent evaporated;

26. The method of claim 25 wherein said metal catalyst comprises titanium dioxide, copper, copper oxide, zinc, zinc oxide, iron, and iron oxide or mixtures thereof.

27. The method of claim 25 wherein said metal catalyst is titanium dioxide.

28. The method of claim 25 wherein said solvent is aqueous.

29. The method of claim 25 wherein said solvent is organic.

30. The method of claim 25 wherein said solvent is a co-solvent mixture of aqueous and organic solvents.

31. The method of claim 25 wherein said metal catalyst is present in said mixture at up to 90% by weight.

32. The method of claim 25 wherein said metal catalyst is present in said mixture is present at concentrations from 20-90%.

33. The method of claim 25 wherein said metal catalyst is present in said mixture is present at concentrations from 50-87%.
